(19) United States
(12) Patent Application Publication
(10) Pub. No.: US 2020/0038047 A1
(43) Pub. Date: Feb. 6, 2020

(54) MEDICAL TREATMENT TOOL

(71) Applicant: OLYMPUS CORPORATION, Tokyo (JP)

(72) Inventor: Masahiro FUJII, Tokyo (JP)

(73) Assignee: OLYMPUS CORPORATION, Tokyo (JP)

(21) Appl. No.: 16/589,331

(22) Filed: Oct. 1, 2019

Related U.S. Application Data
(63) Continuation of application No. PCT/JP2017/014292, filed on Apr. 5, 2017.

(51) Int. Cl. A61B 17/29

(52) U.S. Cl. CPC A61B 17/2909; A61B 2017/294; A61B 2017/2943; A61B 2017/2947

(57) ABSTRACT

A medical treatment tool of the present invention includes a supporting unit; a pair of grasping pieces, at least one of which is supported on the supporting unit so that the at least one grasping piece pivots on a pivot axis orthogonal to a longitudinal axis of the supporting unit; and a driving mechanism causes the grasping piece to pivot. The driving mechanism includes a cylindrical part disposed at a proximal end of the grasping piece and having the pivot axis; a driving member having an outer surface that makes rolling contact with an inner surface of the cylindrical part, the driving member being rotatably supported on an inner side of the cylindrical part so that the driving member rotates about an axis parallel to the pivot axis; and a force transmitting member transmitting a force applied to a proximal end of the supporting unit to rotate the driving member.

### CROSS-REFERENCE TO RELATED APPLICATIONS

This is a continuation of International Application PCT/JP2017/014292, with an international filing date of Apr. 5, 2017, which is hereby incorporated by reference herein in its entirety.

### TECHNICAL FIELD

The present invention relates to a medical treatment tool.

### BACKGROUND ART

There is known a treatment tool in which: one of grasping pieces is pivotally supported on a distal end of a cylindrical supporting unit so as to be pivotable on a pivot axis orthogonal to a longitudinal axis of the supporting unit; an internal gear is disposed on a proximal end side with respect to the aforementioned pivot axis so as to be coaxial with the pivot axis; and a drive gear that meshes with the internal gear is rotated to cause the grasping piece to pivot on the pivot axis so as to close and open the grasping piece (for example, see U.S. Pat. No. 7,699,835).

### SUMMARY OF INVENTION

According to an aspect of the present invention, there is provided a medical treatment tool including a long supporting unit; a pair of grasping pieces, at least one of which is pivotally supported on a distal end of the supporting unit so that the at least one grasping piece can pivot on a pivot axis orthogonal to a longitudinal axis of the supporting unit; and a driving mechanism that causes the grasping piece to pivot. The driving mechanism includes a cylindrical part that is disposed at a proximal end of the grasping piece and has the pivot axis as a center axis; a driving member that has an outer surface that makes rolling contact with an inner surface of the cylindrical part, the driving member being rotatably supported on an inner side of the cylindrical part so that the driving member can rotate about an axis parallel to the pivot axis; and a force transmitting member that transmits a force applied to a proximal end of the supporting unit and thereby rotates the driving member.

### BRIEF DESCRIPTION OF DRAWINGS

FIG. 1 is a perspective view of a distal end portion of a medical treatment tool according to one embodiment of the present invention.

FIG. 2 is an exploded perspective view of the distal end portion of the medical treatment tool illustrated in FIG. 1.

FIG. 3 is a front view illustrating a state in which a fixed grasping piece and a movable grasping piece of the distal end portion of the medical treatment tool illustrated in FIG. 1 are attached.

FIG. 4 is a front view illustrating the relationship between an internal gear provided in the movable grasping piece of the medical treatment tool illustrated in FIG. 1 and an external gear provided in a driving member.

FIG. 5 is a front view illustrating a first modification of the medical treatment tool illustrated in FIG. 1, and illustrates a state in which a fixed grasping piece and a movable grasping piece are attached.

FIG. 6 is a front view illustrating the relationship between an internal gear provided in the movable grasping piece of the medical treatment tool illustrated in FIG. 5 and an external gear provided in a driving member.

FIG. 7 is a front view illustrating a second modification of the medical treatment tool illustrated in FIG. 1.

FIG. 8 is a rear view of the medical treatment tool illustrated in FIG. 7.

FIG. 9 is a rear view of another example of the medical treatment tool illustrated in FIG. 8.

### DESCRIPTION OF EMBODIMENTS

A medical treatment tool 1 according to a first embodiment of the present invention will now be described with reference to the drawings.

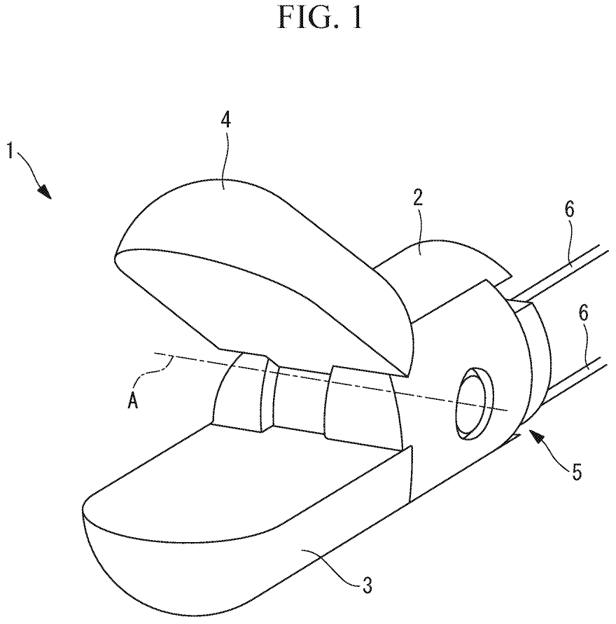

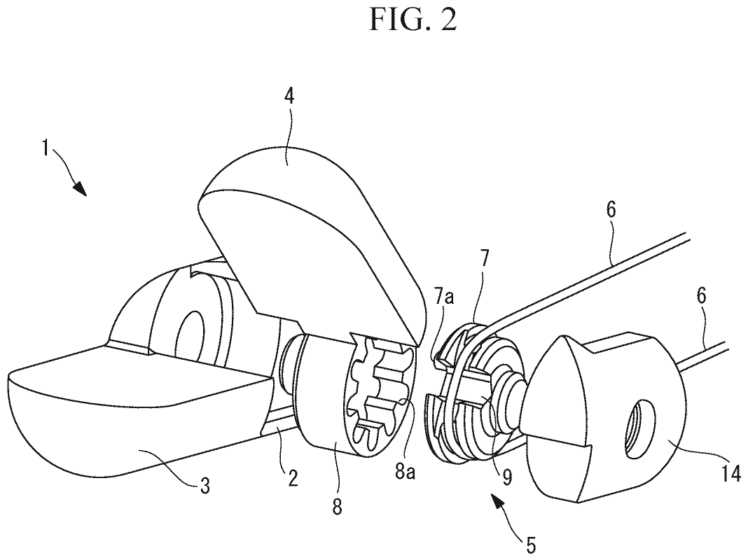

As illustrated in FIGS. 1 and 2, the medical treatment tool 1 of the present embodiment is equipped with a long supporting unit 2 having flexibility, a fixed grasping piece (grasping piece) 3 fixed to a distal end of the supporting unit 2, a movable grasping piece (grasping piece) 4 pivotally supported so as to pivot on a pivot axis A orthogonal to the longitudinal axis of the supporting unit 2 so that the movable grasping piece 4 can open and close relative to the fixed grasping piece 3, and a driving mechanism 5 that causes the movable grasping piece 4 to pivot.

The driving mechanism 5 is equipped with a driving unit (not illustrated), such as a handle or an actuator, that is disposed on a proximal end side of the supporting unit 2 and generates a force, a wire (force transmitting member) 6 that transmits the force generated by the driving unit, a driving member 7 driven by the wire 6 to rotate about an axis parallel to the pivot axis A, and a cylindrical part 8 fixed to the proximal end of the movable grasping piece 4, in which the center axis of the cylindrical part 8 is the pivot axis A. In the drawings, reference sign 14 denotes a cap that rotatably supports the driving member 7.

An internal gear 8a is formed in the inner surface of the cylindrical part 8, and an external gear 7a that meshes with the internal gear 8a so as to be in rolling contact with the internal gear 8a is formed in the outer surface of the driving member 7.

The wire 6, at a lengthwise middle position thereof, is wound around the outer surface of the cylindrical driving member 7, and a fixing member (not illustrated) is fitted into a groove 9 formed in one circumferential position in the driving member 7, and is bonded so as to fix the wire 6 to the driving member 7. In this manner, the wire 6 has two portions flanking the axis of the driving member 7 and extending parallel to each other from the supporting unit 2 toward the proximal end side, and is connected to the driving unit. When one of the two portions of the wire 6 is pulled by the driving unit, the driving member 7 can be rotated in one direction, and when the other portion of the wire 6 is pulled, the driving member 7 can be rotated in a different direction.

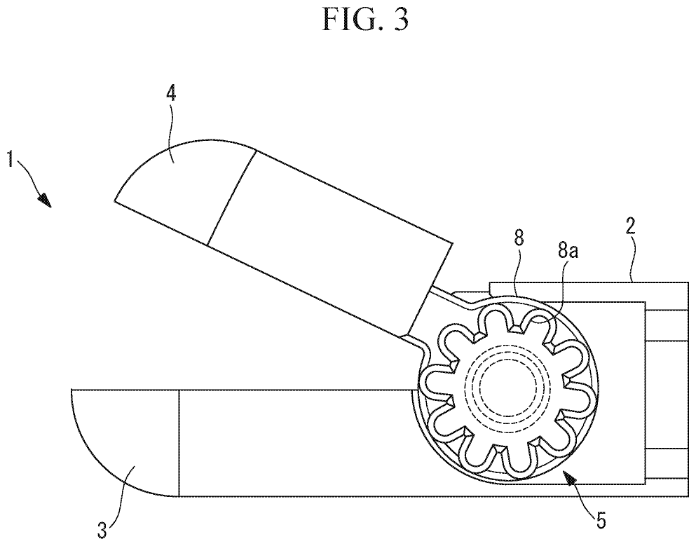

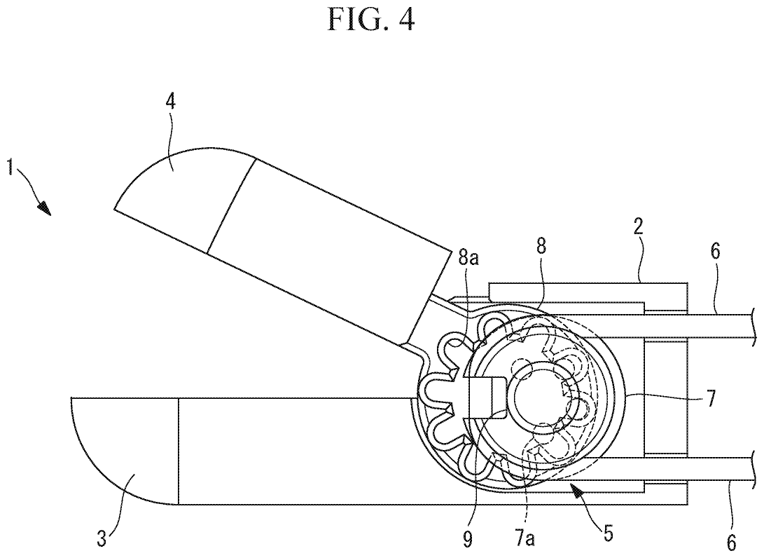

In the present embodiment, as illustrated in FIGS. 3 and 4, the external gear 7a is a pin gear, and the internal gear 8b is a cycloidal gear. The teeth of the pin gear are pins that are equidistant from one another in the circumferential direction except in the region where the groove 9 is formed and the vicinity of the groove 9, and the pins are disposed at four positions along a particular pitch circle radius. The movable grasping piece 4 can securely obtain a pivot angle of 45° or more by using just four pins.

The ratio of the pitch circle radius of the internal gear 8b to the pitch circle radius of the external gear 7a is 2 to 6, and the rotation of the external gear 7a is reduced to ⅙ to ½ and causes the internal gear 8b to rotate.

According to the medical treatment tool 1 configured as such, there is an advantage in that the movable grasping piece 4 can be caused to pivot at a torque two to six times greater than the torque generated by the force applied to the wire 6 by the driving unit, and a treatment subject site or the like can be securely grasped between the movable grasping piece 4 and the fixed grasping piece 3 with a small force.

In addition, in the present embodiment, the movable grasping piece 4 that is caused to pivot on the pivot axis A is caused to pivot by driving and rotating the cylindrical part 8 fixed to the proximal end portion, and the center axis of the cylindrical part 8 is the pivot axis A of the movable grasping piece 4; thus, when the movable grasping piece 4 pivots, the dimension in the radial direction about the pivot axis A does not change, and, unlike in the related art, the proximal end portion of the grasping piece does not significantly protrude in the radial direction of the supporting unit 2.

This medical treatment tool 1 is, for example, delivered to the inside of the body via a channel of an endoscope or a channel of an overtube; thus, naturally, during insertion into the body, the movable grasping piece 4 is closed relative to the fixed grasping piece 3 so as to reduce the diameter of the overall structure and make insertion smooth. When the treatment subject site is treated while having the medical treatment tool 1 of the present embodiment protruding from the distal end of the channel of an endoscope or the like, the treatment is performed by opening and closing the movable grasping piece 4 relative to the fixed grasping piece 3.

This feature is advantageous in that, if, for some reason, the entire medical treatment tool 1 is withdrawn into the channel while the movable grasping piece 4 is still in an open state relative to the fixed grasping piece 3, the proximal end portion of the movable grasping piece 4 in a wide open state does not get stuck at the opening of the channel and thus the withdrawal can be carried out smoothly. This is because the outer diameter of the proximal end portion of the movable grasping piece 4 of the medical treatment tool 1 of the present embodiment remains the same irrespective of whether the movable grasping piece 4 is open or closed. As a result, breaking of the medical treatment tool 1 can be prevented.

Furthermore, in the medical treatment tool 1 of the present embodiment, the internal gear 8a and the external gear 7a are constituted by a pin gear and a cycloidal gear. Thus, there is an advantage in that the structure of the section used to transmit a force from the driving member 7 to the movable grasping piece 4 can be simplified.

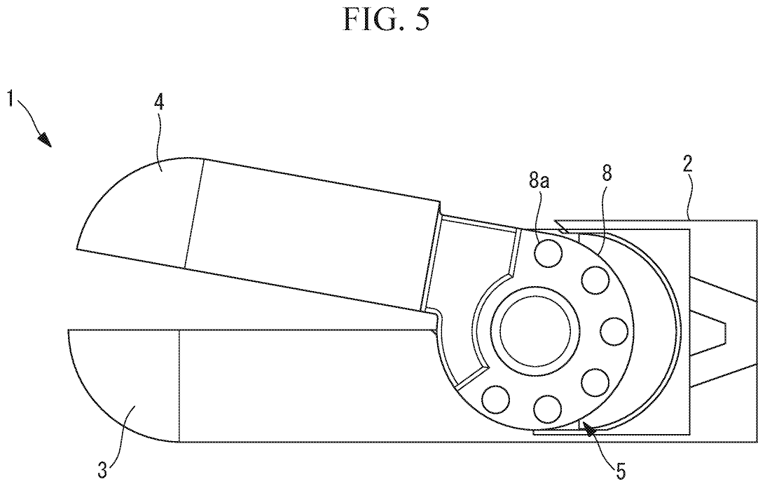

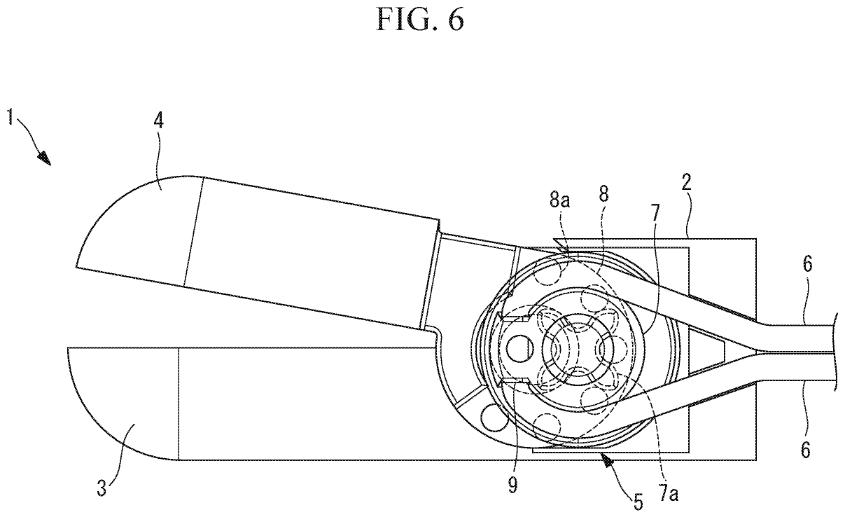

As illustrated in FIGS. 5 and 6, alternatively, the internal gear 8a may be constituted by a pin gear and the external gear 7a may be constituted by a cycloidal gear.

Alternatively, other types of gears may be employed instead of the pin gear and the cycloidal gear.

In the present embodiment, the force is transmitted from the driving member 7 to the movable grasping piece 4 through rolling contact achieved by meshing between the internal gear 8a and the external gear 7a; alternatively, the force may be transmitted through friction contact between the inner surface of the cylindrical part 8 provided in the movable grasping piece 4 and the outer surface of a column provided on the driving member 7. In this case also, by setting the diameter of the inner surface of the cylindrical part 8 to be sufficiently larger than the diameter of the outer surface of the column, the rotation of the driving member 7 is reduced and transmitted to the movable grasping piece 4.

In the present embodiment, the grasping piece 3 is fixed and the grasping piece 4 is caused to pivot. Alternatively, both grasping pieces 4a and 4b may pivot so that the grasping pieces 4a and 4b can open and close in synchronization.

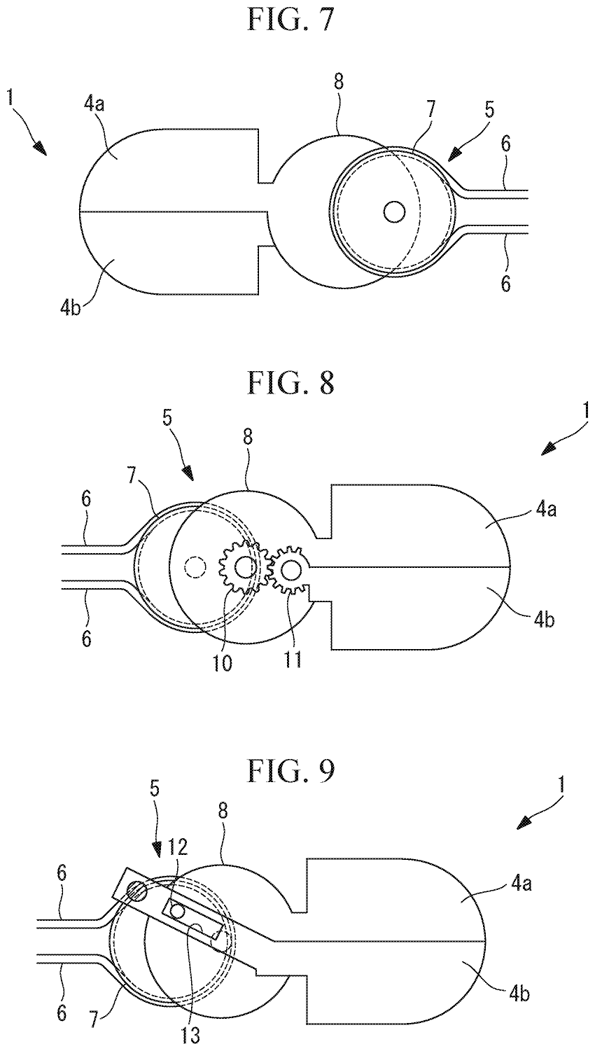

For example, as illustrated in FIGS. 7 and 8, a gear 10 may be disposed on a surface of the cylindrical part 8 at the proximal end of the grasping piece 4a, the surface being on the axially opposite side of the internal gear 8a, and a gear 11 that meshes with the gear 10 may be disposed at the proximal end of the other grasping piece 4b. As a result, when the wire 6 is pulled to cause the grasping piece 4a to pivot in one direction, the other grasping piece 4b is caused to pivot in an opposite direction via the gear 11 meshed with the gear 10 provided in the grasping piece 4a, and, thus, the pair of grasping pieces 4a and 4b can be opened and closed.

As illustrated in FIG. 9, instead of the gears 10 and 11, a pin 12 that protrudes from the cylindrical part 8 of the grasping piece 4a toward the side opposite to the internal gear 8a may be provided, the other grasping piece 4b may be pivotally attached to the supporting unit 2, and a cam groove 13 that guides the pin 12 may be formed in the grasping piece 4b.

In this manner, when the grasping piece 4a is caused to pivot in one direction by the driving member 7, the position of the pin 12 in the cylindrical part 8 changes. Thus, by moving the pin 12 within the cam groove 13, the other grasping piece 4b can also be caused to pivot in a different direction, and the pair of grasping pieces 4a and 4b can be simultaneously opened and closed in synchronization.

Although a long supporting unit 2 having flexibility is described as one example, a rigid supporting unit 2 may be employed instead.

As a result, the following aspect is read from the above described embodiment of the present invention.

According to an aspect of the present invention, there is provided a medical treatment tool including a long supporting unit; a pair of grasping pieces, at least one of which is pivotally supported on a distal end of the supporting unit so that the at least one grasping piece can pivot on a pivot axis orthogonal to a longitudinal axis of the supporting unit; and a driving mechanism that causes the grasping piece to pivot. The driving mechanism includes a cylindrical part that is disposed at a proximal end of the grasping piece and has the pivot axis as a center axis; a driving member that has an outer surface that makes rolling contact with an inner surface of the cylindrical part, the driving member being rotatably supported on an inner side of the cylindrical part so that the driving member can rotate about an axis parallel to the pivot axis; and a force transmitting member that transmits a force applied to a proximal end of the supporting unit and thereby rotates the driving member.

According to this aspect, the force applied to the proximal end of the supporting unit is transmitted to the distal end of the supporting unit through the force transmitting member and causes the driving member to rotate. As a result, the outer surface of the driving member is brought into rolling contact with the inner surface of the cylindrical part formed at the proximal end of the grasping piece, the rotative force of the driving member is transmitted to the cylindrical part, and the pair of grasping pieces open and close as they pivot on the pivot axis. As a result, the treatment subject site can be grasped or can be subjected to a treatment such as pressing and spreading.

In this case, since the center axis of the cylindrical part that rotates when the grasping piece is caused to pivot is the pivot axis of the grasping piece, the dimension in the radial direction of the cylindrical part does not change despite the pivoting of the grasping piece, and unlike in the related art, the proximal end portion of the grasping piece does not extensively protrude in the radial direction of the supporting unit. As a result, even when the treatment tool is moved toward the proximal end side while the grasping pieces are in a wide open state, the proximal end portion of the grasping piece does not interfere with surroundings, and the treatment tool can move smoothly.

In the aspect described above, an internal gear may be formed in the inner surface of the cylindrical part, and an external gear that meshes with the internal gear may be formed in an outer surface of the driving member.

In this manner, meshing between the external gear and the internal gear enables the rotative force of the driving member to unfailingly be transmitted to the grasping piece, and, thus, the grasping force or the force of pressing and spreading the treatment subject site applied by the grasping piece can be improved.

In the aspect described above, one of the internal gear and the external gear may be a pin gear, and the other may be a cycloidal gear.

In this manner, a driving mechanism that unfailingly transmits a force can be configured by using a pin gear having a simple structure.

In the aspect described above, the supporting member may have flexibility.

In this manner, the supporting unit can be inserted along a bending path by bending the supporting unit, and the pair of grasping pieces can be delivered to the desired treatment subject site.

### REFERENCE SIGNS LIST

1 medical treatment tool

2 supporting unit

3 fixed grasping piece (grasping piece)

4 movable grasping piece (grasping piece)

4a, 4b grasping piece

5 driving mechanism

6 wire (force transmitting member)

7 driving member

7a external gear

8 cylindrical part

8a internal gear

A pivot axis

1. A medical treatment tool comprising:
a long supporting unit;
a pair of grasping pieces, at least one of which is pivotally supported on a distal end of the supporting unit so that the at least one grasping piece pivots on a pivot axis orthogonal to a longitudinal axis of the supporting unit; and
a driving mechanism that is configured to cause the grasping piece to pivot,
wherein the driving mechanism includes a cylindrical part that is disposed at a proximal end of the grasping piece and has the pivot axis as a center axis; a driving member that has an outer surface that is configured to make rolling contact with an inner surface of the cylindrical part, the driving member being rotatably supported on an inner side of the cylindrical part so that the driving member can rotate about an axis parallel to the pivot axis; and a force transmitting member that transmits a force applied to a proximal end of the supporting unit and thereby rotates the driving member.

2. The medical treatment tool according to claim 1, wherein an internal gear is formed in the inner surface of the cylindrical part, and
an external gear that meshes with the internal gear is formed in an outer surface of the driving member.

3. The medical treatment tool according to claim 2, wherein one of the internal gear and the external gear is a pin gear, and the other is a cycloidal gear.

4. The medical treatment tool according to claim 1, wherein the supporting unit has flexibility.
